(19) United States
(12) Patent Application Publication
(10) Pub. No.: US 2003/0232023 A1
(43) Pub. Date: Dec. 18, 2003

(54) Oral hygiene system and method

(76) Inventor: Michael Arnold, Irvine, CA (US)

Correspondence Address: Eric K. Satermo, P.O. Box 19099, Irvine, CA, 92623-9099, US

(21) Appl. No.: 10/172,366

(22) Filed: Jun. 14, 2002

(51) Int. Cl. A61K 7/20; A61C 5/00

(52) U.S. Cl. 424/53; 433/217.1

(57) ABSTRACT

An oral hygiene system includes a whitening solution, and applicator, and a rinse. The whitening solution includes a concentration of hydrogen peroxide for whitening teeth within a predetermined period of time without the use of a mouthpiece. The applicator may include a resilient head capable of conforming to the topography of a tooth surface, and may be used for applying gelatinous whitening solution to teeth. The rinse is configured to reduce organic sulfides in the mouth, and may include zinc acetate. The whitening solution may be applied to the teeth for predetermined amount of time, for example, less than about 10 minutes. The solution may then be rinsed off with the rinse. The procedure may then be repeated periodically, for example, daily until the desired shade of whiteness is achieved.

### BACKGROUND OF THE INVENTION

1. Field of the Invention

The present invention relates to oral cosmetics dental hygiene products and, more particularly, to solutions and systems for bleaching or whitening teeth and for treating halitosis, and to associated methodology.

2. Description of the Related Art

Proper oral hygiene is practiced daily. Typically oral hygiene procedures include tooth brushing with an abrasive compound (i.e., toothpaste), flossing, and rinsing with a mouthwash. Although useful for maintaining good oral hygiene, such procedures are unable to whiten the dentin adequately and to freshen of the breath in cases of severe halitosis.

Conventional tooth whitening is accomplished by using hydrogen peroxide on the dentin, which is the main, calcareous part of a tooth, beneath the enamel and surrounding the pulp chamber and root canals. Conventional tooth-whitening solutions include gel forms of hydrogen peroxide and hydrogen peroxide derivatives such as carbamide peroxide. These compositions are typically applied in conjunction with a mouthpiece or tray that fits snuggly over the surface of the teeth. The tooth-whitening process takes many hours to achieve desired results. Drawbacks including discomfort and potential injury due to poorly fitting trays and long duration for minimal efficacy.

Halitosis is a major concern for many people. Halitosis originates primarily from excrement of certain oral bacteria. Conventional mouthwashes typically mask oral malodor. One of the shortcomings of mouthwashes is that they are usually not bactericidal and, therefore, do not address the bacterial cause of halitosis.

In addition to the deficiencies in each respective procedure (i.e., tooth whitening and breath freshening), neither procedure provides a complete solution for oral hygiene.

### BRIEF SUMMARY OF THE INVENTION

An oral hygiene system includes a whitening solution, and applicator, and a rinse. The whitening solution includes a concentration of hydrogen peroxide for whitening teeth within a predetermined period of time without the use of a mouthpiece. The applicator may include a resilient head capable of conforming to the topography of a tooth surface, and may be used for applying gelatinous whitening solution to teeth. The rinse is configured to reduce organic sulfides in the mouth, and may include zinc acetate.

The whitening solution may be applied to the teeth for predetermined amount of time, for example, less than about 10 minutes. The solution may then be rinsed off with the rinse. The procedure may then be repeated periodically, for example, daily until the desired shade of whiteness is achieved.

Other features and advantages of the present invention will become apparent to those skilled in the art from a consideration of the following detailed description taken in conjunction with the accompanying drawings.

### BRIEF DESCRIPTION OF THE SEVERAL VIEWS OF THE DRAWINGS

FIG. 1 is a perspective view of a oral hygiene kit;

FIG. 2 is a cross-sectional view of an applicator and a tooth; and

FIG. 3 is a flowchart illustrating oral hygiene methodology.

### DETAILED DESCRIPTION OF THE INVENTION

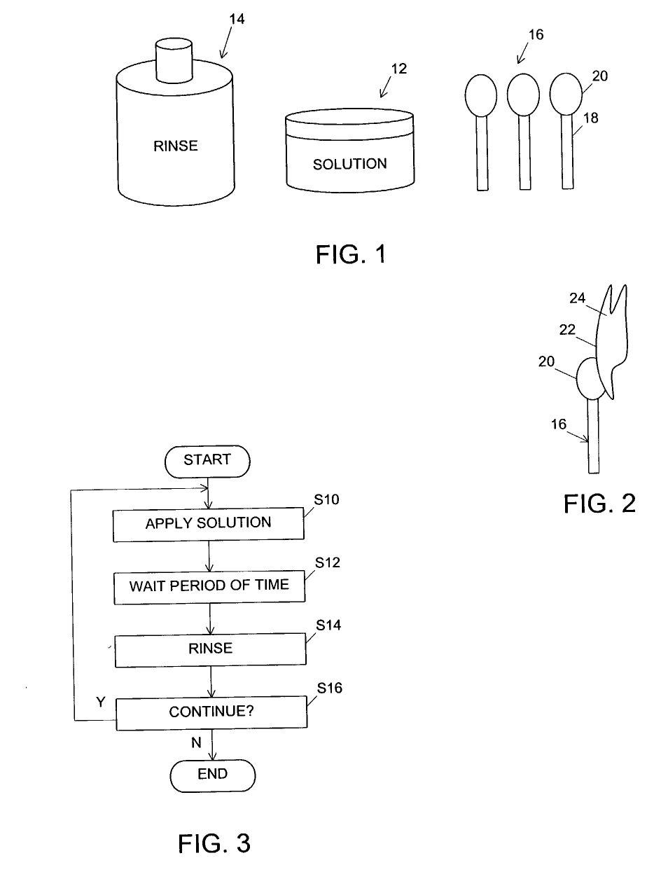

Referring to the drawings, an oral hygiene system 10 is illustrated in FIG. 1 and may include a whitening solution 12 and a rinse 14. Exemplary system 10 may also include one or more applicators 16 for applying the solution 12 to the teeth. The system 10 may be distributed in the form of a kit, with the whitening solution 12 and the mouthwash 14 packaged in respective containers.

In a number of embodiments, the whitening solution 12 may include hydrogen peroxide that is capable of whitening the enamel of teeth within a predetermined period of time. For example, the whitening solution 12 may include at least about a 5% concentration of hydrogen peroxide, and may include about a 10% concentration of hydrogen peroxide.

Although conventional mouthpieces or trays may be used, in a number of embodiments, for example, for a gelatinous whitening solution, the applicator 16 is employed to apply the whitening solution 12 to teeth. Accordingly, the whitening solution 12 is applicable without the use of mouthpieces or trays. The applicator 16 may include a handle 18 with a head 20. A number of embodiments of the applicator 16 include a resilient head capable of conforming to the topography of the surface 22 of a tooth 24, as shown in FIG. 2. For example, the head 20 may include foam or fibrous material capable of deformation, either elastic or substantially inelastic deformation.

In a number of embodiments, the whitening solution 12 may be viscous or gelatinous. Accordingly, the applicator 16 may be used to “scoop up” an amount of the solution 12 on the head 20 for application to the teeth, without the solution 12 running off or being absorbed by the head 20 like a liquid. As shown examples below, a number of embodiments utilize hydrogen peroxide in the whitening solution 12.

Regarding the rinse 14, in some of the embodiments the rinse 14 is capable of reducing organic sulfides on the surfaces within the mouth, including the surfaces of soft tissue such as the tongue and gingivae and the surfaces of hard tissue such as the enamel of the teeth. In a number of embodiments, the rinse 14 includes zinc or copper salt solutions.

Referring to FIG. 3, a user may apply the whitening solution 14 to the teeth (step S10). The user may then wait for a period of time (step S12), for example, less than 10 minutes. The user may then remove the solution 12 from the mouth by rinsing with the rinse 14 (step S14). To whiten the teeth further (step S16), the user may repeat the procedure any number of times. For example, the procedure may be repeated daily for an extended period of time, e.g., one week or two weeks.

Examples of embodiments of the oral hygiene system 10 follow.

### EXAMPLE 1

In a number of embodiments, the whitening solution 12 is an aqueous hydrogen peroxide gel composition including the following ingredients:

| Ingredient | Amount (%) |
|---|---|
| hydrogen peroxide | 10 |
| purified water | 88 |
| carbomer 940 | 1 |
| triethanol amine | 1 |

In a number of embodiments, the rinse 14 is a mouthwash composition including the following ingredients:

| Ingredient | Amount (%) |
|---|---|
| zinc acetate | 1.0 |
| purified water | 90.6 |
| ethanol | 6.0 |
| menthol | 0.2 |
| sucralose | 0.1 |
| polysorbate | 2.0 |
| oil of peppermint | 0.1 |

The gelatinous whitening solution 12 may be applied to the surface of the teeth that the user desires to whiten. After applying, the user may wait for a period of time, or an application time; for example, the application time of the gelatinous solution 12 may be up about 10 minutes. For example, in a number of embodiments, the application time may range from about 1 minute to about 5 minutes. In other embodiments, the application time may range from about 2 minutes to about 3 minutes.

The rinse 14 may be applied by rinsing up to about 2 minutes. For example, in some embodiments, the application time of the rinse 14 may range from about 10 seconds to about 45 seconds. In other embodiments, the application time may be about 30 seconds.

### EXAMPLE 2

In a number of embodiments, the whitening solution 12 is an aqueous hydrogen peroxide gel composition including the following ingredients:

| Ingredient | Amount (grams) |
|---|---|
| deionized water | 660 |
| hydrogen peroxide (35%) | 286 |
| carbopol 934 | 20 |
| triethanol amine | 10 |

In this example, the concentration of hydrogen peroxide in the whitening solution 12 is about 30%.

### EXAMPLE 3

In a number of manufacturing embodiments for the whitening solution 12, all lines and surfaces may first be pacivated for at least 24 hours. Carbopol may be slowly added to warm water (e.g., 50° C.) with adequate stirring or homogenization until a uniform suspension is formed. The solution of carbopol and water may then be cooled to about 35° C. Peroxide may then be added to the cooled solution, with the resulting solution being stirred for about 10 minutes. Triethanol amine may then be added as a 1:1 solution in deionized water, with the resulting solution being stirred until a homogeneous gel is obtained.

### EXAMPLE 4

In a number of embodiments, the rinse 14 may include the following ingredients:

| Ingredient | Amount (grams) |
|---|---|
| Deionized Water | |
| Ethanol | 1,000 |
| polysorbate 20 | 450 |
| methyl paraben | 2 |
| 1-menthol crystals (ground) | 15 |
| aspartame | 25 |
| zinc acetate | 118 |
| spearmint | 18 |

To produce, zinc acetate, sucralose, and ethanol may be added to warm water (e.g., 50° C.). This solution may then be stirred until homogeneous. A homogeneous solution of polysorbate 20, menthol, methyl paraben, and spearmint may then be added to the solution of zinc acetate, sucralose, ethanol, and water. The resulting solution may be stirred to homogeneity while cooling. The aqueous solutions of zinc (or copper) salts may reduce oral malodor by chemically modifying the composition of the aerosol or gas that comprises the breath, so that many of the malodiferous agents, typically organic sulfides, are not present, thus yielding fresh breath.

### EXAMPLE 5

In a number of embodiments, the rinse 14 may be an aqueous hydrogen peroxide rinse including the following ingredients:

| Ingredient | Amount (%) |
|---|---|
| hydrogen peroxide (35%) | 8.75 |
| deionized water | 81.62 |
| ethanol | 8.00 |
| Tween 60 | 0.61 |
| 1-menthol crystals | 0.3 |
| eucalyptus oil | 0.05 |
| camphor | 0.02 |
| sucralose | 0.3 |
| spearmint | 0.175 |
| peppermint | 0.175 |

To produce, all lines and surfaces may be pacivated for at least 24 hours. Sucrolose and ethanol may then be added to warm water (e.g., 50° C.), with this solution then being stirred until homogeneous. A homogeneous solution of Tween 60, menthol, eucalyptus oil, and camphor may then be added to the solution of sucrolose, ethanol, and water. The resulting solution may then be stirred to homogeneity while cooling, for example, to about 37° C. Hydrogen peroxide may then be added, with the resulting solution cooled to ambient temperature.

### EXAMPLE 6

As an application example, the whitening solution 12 may be applied directly to the surface of the teeth that the user desires a whiter color. The user may then leave the solution 12 on the teeth for a period of time, or application time, for example, from 1 minute to 10 minutes. The application time may also range from 1.5 minutes to 5 minutes, or from 2 minutes to 3 minutes. The rinse 14 may then be applied by rinsing for up to about 120 seconds. The rinse time may also range from about 10 seconds to 45 seconds, or for about 30 seconds. The rinse 14 may then be expulsed from the mouth.

The procedure may then be repeated. For example, the user may repeat the procedure after a period of time, for example, 12 hours. In other embodiments, the user may repeat the procedure daily. The daily repetition may continue for up to a month. In other embodiments, the daily repetition may continue for about 14 days.

After the desired whiteness of the teeth is achieved, the user may utilize the rinse 14 on a daily basis to control halitosis. For example, from about 0.2 oz to about 3 oz of rinse 14 may be used. In other embodiments, about 0.5 oz to about 2 oz, e.g., about 1 oz, of rinse 14 may be used for a period up to about 120 seconds.

Those skilled in the art will understand that the preceding exemplary embodiments of the present invention provide the foundation for numerous alternatives and modifications thereto. For example, although the methods, solutions, and apparatus have been described in relation to teeth, the description is equally applicable to dental prosthetics such as dentures and crowns and to oral appliances. These other modifications are also within the scope of the present invention. Accordingly, the present invention is not limited to that precisely as shown and described in the present invention.

What is claimed is:

1. An oral hygiene kit comprising:
a whitening solution for whitening teeth within a predetermined period of time without the use of a mouthpiece;
an applicator including a resilient head for conforming to the topography of a tooth surface, the applicator for use in applying the whitening solution to teeth; and
a rinse for reducing organic sulfides.

2. A kit as claimed in claim 1 wherein the predetermined amount of time is less than about 10 minutes.

3. A kit as claimed in claim 1 wherein the predetermined amount of time is less than about 3 minutes.

4. A kit as claimed in claim 1 wherein the head of the applicator includes foam.

5. A kit as claimed in claim 1 wherein the whitening solution is gelatinous.

6. A kit as claimed in claim 1 wherein the whitening solution includes hydrogen peroxide.

7. A kit as claimed in claim 1 wherein the rinse includes zinc acetate.

8. An oral hygiene method comprising:
applying a whitening solution to teeth without the use of a tray, the whitening solution capable of bleaching tooth enamel;
waiting a period of time;
rinsing the whitening solution from the teeth.

9. A method as claimed in claim 8 wherein the applying step comprises applying the whitening solution with an applicator.

10. A method as claimed in claim 9 wherein the applicator includes a resilient head capable of conforming to the topography of teeth.

11. A method as claimed in claim 8 wherein the waiting step comprises waiting a period of time less than about 10 minutes.

12. A method as claimed in claim 8 wherein the waiting step comprises waiting a period of time less than about 3 minutes.

13. A method as claimed in claim 8 further comprising repeating the applying, waiting, and rinsing steps about 12 hours later.

14. A method as claimed in claim 8 further comprising repeating the applying, waiting, and rinsing steps daily.

15. A method as claimed in claim 8 wherein the applying step comprising applying a gelatinous whitening solution.

16. A method as claimed in claim 8 wherein the applying step comprises applying a whitening solution including hydrogen peroxide.

17. A method as claimed in claim 8 wherein the rinsing step comprises rinsing with a mouthwash including zinc acetate.

18. A dental whitening solution comprising about a 10% concentration of hydrogen peroxide.

19. A solution as claimed in claim 18 wherein the solution is gelatinous.

20. A solution as claimed in claim 18 wherein the whitening solution includes the following ingredients and concentrations:

| | |
|---|---|
| hydrogen peroxide | 10% |
| purified water | 88% |
| carbomer 940 | 1% |
| triethanol amine | 1% |
